(19) United States
(12) Patent Application Publication
(10) Pub. No.: US 2024/0082197 A1
(43) Pub. Date: Mar. 14, 2024

(54) DELIVERY METHODS FOR OMEGA-3'S AND COMPOSITIONS FOR VITAMINS AND MINERALS USED TO ENHANCE VISUAL ACUITY AND MENTAL DEVELOPMENT IN THE HUMAN BODY

(71) Applicant: Viva Life Science, Inc., Costa Mesa, CA (US)

(72) Inventors: Thuong Cao Do, Westminster, CA (US); Ping Yang, Fountain Valley, CA (US)

(21) Appl. No.: 18/369,616

(22) Filed: Sep. 18, 2023

Related U.S. Application Data
(63) Continuation of application No. 17/229,693, filed on Apr. 13, 2021, now abandoned.
(60) Provisional application No. 63/009,279, filed on Apr. 13, 2020.

(51) Int. Cl. A61K 31/202; A23L 33/105; A23L 33/12; A23L 33/125; A23L 33/14; A23L 33/155; A23L 33/16; A61K 9/00; A61K 9/48; A61K 9/50; A61K 31/047; A61K 31/07; A61K 31/197; A61K 31/355; A61K 31/375; A61K 31/4188; A61K 31/4415; A61K 31/455; A61K 31/51; A61K 31/519; A61K 31/525; A61K 31/593; A61K 31/685; A61K 31/714; A61K 33/00; A61K 33/08; A61K 33/18; A61K 33/26; A61K 33/30; A61K 35/60; A61K 36/064; A61K 36/28

(52) U.S. Cl. CPC A61K 31/202; A23L 33/105; A23L 33/12; A23L 33/125; A23L 33/14; A23L 33/155; A23L 33/16; A61K 9/0053; A61K 9/4858; A61K 9/50; A61K 31/047; A61K 31/07; A61K 31/197; A61K 31/355; A61K 31/375; A61K 31/4188; A61K 31/4415; A61K 31/455; A61K 31/51; A61K 31/519; A61K 31/525; A61K 31/593; A61K 31/685; A61K 31/714; A61K 33/00; A61K 33/08; A61K 33/18; A61K 33/26; A61K 33/30; A61K 35/60; A61K 36/064; A61K 36/28; A23V 2002/00

(57) ABSTRACT

The present invention relates to the delivery method of Omega-3's and nutritional supplements for the human diet used to aid mental and visual development of human embryos as well as enhance visual acuity and mental fitness in humans. The invention contains vitamins, minerals, and microencapsulated fish oil powder containing DHA and EPA.

### CROSS REFERENCE TO RELATED APPLICATIONS

This application is a continuation of U.S. Utility application Ser. No. 17/229,693, filed on Apr. 13, 2021, which is related to and claims priority of U.S. provisional application No. 63/009,279, filed on Apr. 13, 2020. The Applications are hereby incorporated by reference in their entireties. The priority of these applications are expressly claimed, and the disclosures are hereby incorporated by reference in their entirety.

### FIELD OF THE INVENTION

The present invention relates to the delivery method of Omega-3's for the human diet used to enhance visual acuity and mental development while maintaining eye and neural health in the human body.

### BACKGROUND OF THE INVENTION

With today's busy/modern lifestyle and a need for nutritional convenience, many have gone beyond the multivitamin and resorted to the complete nutritional beverage such that contains a combination of nutrients that is made for ready delivery of a range of nutritional substances meant to provide functional meal replacement. A need for this type of convenient, on-the-go nutrition has led to the development of many products that are meant to contain a complete profile of amino acids, vitamins, minerals and lipids to sustain and maintain cellular replication and overall general health. Many of these supplements and food products are not very appetizing or appealing due to certain odors, and unpleasant properties, and the usual consistencies that affect the mouth feel of the product as well as the taste perception.

An excellent example would be the consumption of fish oil for intake of Omega-3 fatty acids. These fatty acids are essential to the development and maintenance of cells throughout the body. Numerous Omega-3 products exist in the market place today, but many fall short in the categories of taste/flavor and convenience. The combination of taste/favor and convenience are important beyond the mere ability of a product to be transiently pleasant upon consumption. Studies have shown adherence to medication plans set up by their doctors depend on eligibility of the medication. Many studies show incidence of medication adherence by patients even when the medications are critical to sustain health. This phenomenon also acts together with supplement adherence where the patient begins taking supplement necessary to achieve better health, yet discontinues the medication for takes sufficient amounts to achieve the nutritional or medical goal therapy due to poor palatability of the supplements.

Two major factors for supplement adherence are dosage forms and overall taste. Many supplements culminate on that is physically too large for the average consumer to consumer or otherwise inconvenient for example, a supplement bearing label instructions to consume two tablets with a meal may contain a total quantity of the combination of ingredients that exceeds 1.4 grams per tablet. When a supplement in tablet form ways more than 1 gram, the large size makes the tablet unpalatable. Similarly, to achieve same quantity of delivery to the patient, the label would recommend, as many supplements to, the consumption of six to eight tablets in one sitting. That is the similarly inconvenience leads to poor patient compliance.

Taste is a big factor in supplement adherence. Many supplements have unpleasant odors and flavors, including specifically fish oil supplements. Flavor supplements have limited effectiveness, particularly where the underlying composition has strong odor and taste characteristics. Attempts to use aromatic fruit flavoring such as lemon to mask the taste and perception of products containing fish oils are notoriously unsuccessful. Packaging and formulation strategies, such as gelatin encapsulation, micro capsules, liposomal encapsulation's and similar strategies are inherently limited.

### PRIOR ART

Many studies have focused on the use of omega 3's, specifically DHA and EPA, for use in general health as well as developmental health. Traditionally, these nutrients are obtained by consuming foods such as salmon, sardines, mackerel and other omega-3 rich foods. The supplement industry has introduced other means of obtaining these nutrients through the introduction of fish oils and fish oils encapsulated with gelatin. While these means of obtaining omega-3 are sufficient for some, for the majority of consumers, the odor of the fish oil is so unappealing that the consumer may forego the health benefits due to the unpleasant taste and smell. Although the introduction of gelatin encapsulated fish oils, has reduced the odors, fish odor still remains.

Other forms of microencapsulated algae containing DHA have been in the market for a few years, yet manufacturers have still not been able to provide an odor free product. There are also strict environmental conditions for gelatin encapsulated fish oils. If the temperature is above the stated range, the gelatin capsule may deform or even burst, spilling out the contents of the gelatin capsule. There is also an inconvenience with the use of fish oils in a bottle. Most of the time the contents of the fish oil would have to be poured out into a measuring device each time a dosage is to be consumed. This may lead to dosage errors, where too much or too little of the required daily dosage would not be obtained, as well as exposing the oil to the environment many times, leading to oxidation and rancidity issues.

### SUMMARY OF THE INVENTION

The introduction of microencapsulated fish oil provides DHA and EPA in a powder form that has negligible fish odor. With the use of this microencapsulated fish oil powder, omega-3 nutritional supplement compliance and adherence will be dramatically increased. Additionally, by combining vitamins, minerals, microencapsulated fish oil powder containing DHA and EPA, this invention may be orally administered as directed to obtain nutritional and health benefits. By using a microencapsulated fish oil powder in this invention, the unpleasant odor that reduces patient compliance with consuming an adequate amount of the beneficial Omega-3 compounds is eliminated. A nutritionally or therapeutically effective dosage is easily administered, together with other beneficial substances, and the manufacturing, storage, and environmental conditions have little impact on the end product.

The invention of this nutritional supplement combination may be used to supplement deficiencies in the diet as well as provide beneficial health effects. The recitation of a specific identified set of compositions grouped within either of the defined Composition A or Composition B is open-ended and is not meant to preclude the addition of additional compositions within the scope of either of Composition A or Composition B, including combinations thereof within either set and substitutions for synonyms, analogues, or derivatives thereof as described herein.

A healthy lifestyle has become particularly popular in recent years and the focus on nutritional benefits has extended to our future children. Prenatal multivitamins have become popular with expectant mothers and fathers, but many lack important nutrients for gestational development as well as postnatal development. As mentioned earlier the drawback of many supplement products on the market is actually taking these supplements daily and maintaining that nutritional plan. By providing a palatable formula for administration, the present invention encourages delivery of a nutritionally or therapeutically effective balance of the compounds listed below.

There are many products that focus on either brain health or eye health, but not both. With the invention of this nutritional supplement many health benefits can be obtained for both brain and eye health as well as address other short falls with other supplement products in the market.

### DETAILED DESCRIPTION OF THE INVENTION

The present invention is compositions and methods enabling the precise delivery of DHA and EPA compositions with no perceivable odor that is if nutritional supplements formulated pursuant to the invention aid in the mental and visual development of human embryos, supplement nutritional deficiencies, and may improve visual acuity and mental cognition in the human mammal. Although the invention is described in the context of a list of potential ingredients, individual embodiments of the invention may be formulated with sub-sets of the compounds and compositions listed below, as long as the particular formulations do not depart from the spirit of the invention providing an advantageous form of Omega 3 compositions in a therapeutic or nutritional composition, dosage, and following administration.

The present invention may include vitamin A. Vitamin A is involved in physiological processes that result in cellular differentiation, cellular maturity, and cellular specificity. In one embodiment of the present invention, vitamin A may be in a form that is a precursor (pro-vitamin) or metabolite of vitamin A that provides similar nutritional value as vitamin A. For example, the pro-vitamin A carotenoid, may be beta carotene. Beta carotene is converted to other forms of vitamin A, specifically retinol, within the body as needed, thereby avoiding the risk of retinol toxicity. Mayne, FASEB J 10:690-701 (1996). In a specific embodiment, vitamin A may be in one or more of the forms of retinol acetate (also known as retinyl acetate or vitamin A acetate), retinol (vitamin A alcohol), retinol palmitate (also known as retinyl palmitate or vitamin A palmitate), retinoic acid (tretinoin), retinal, beta-cryptoxanthin, alpha-carotene, beta-carotene, gamma-carotene, and provitamin A carotenoids.

Vitamin A may be in the form of beta carotene as beta carotene also has powerful anti-oxidant properties. Antioxidants are important during physiologically stressful events for numerous reasons.

In a specific embodiment of the compositions, kits and methods of the present invention, vitamin A may be included in amounts ranging from about 550 IU to about 1650 IU. In another specific embodiment, vitamin A in the form of Vitamin A Palmitate may be included in amounts ranging from about 800 IU to about 900 IU.

The compositions, kits and methods of the present invention may comprise or use one or more B-complex vitamins. This class of vitamins comprises water-soluble nutrients generally not stored in the body. They play roles in a variety of biological processes critical to the health of pregnant women, lactating women, and fetuses such as, for example, the metabolism of homocysteine. The B-complex vitamins that may be included in the compositions, kits and methods of the present invention comprise one or more of vitamin B1, vitamin B2, vitamin B3, vitamin, B5, vitamin B6, and vitamin B12.

The compositions, kits and methods of the present invention may comprise or use vitamin B1. Vitamin B1 plays a role in carbohydrate metabolism and neural function. It is a coenzyme for the oxidative decarboxylation of alpha-ketoacids (e.g., alpha-ketoglutarate and pyruvate) and for transketolase, which is a component of the pentose phosphate pathway. NATIONAL RESEARCH COUNCIL, RECOMMENDED DIETARY ALLOWANCES, page 125 (10th ed. 1989) (hereinafter “RDA”). In one embodiment, vitamin B1 may be in one or more of the forms of thiamine, thiamine monophosphate, thiamine diphosphate, thiamine triphosphate, acetiamine, allithiamine, prosultiamine and S-acyl derivatives of thiamine such as benfotiamine, fursultiamine and salts and esters thereof. In one embodiment, vitamin B1 as Thiamine Hydrochloride is included in amounts ranging from about 0.1 to 2.0, 0.25-0.75 and specifically 0.4 mg to about 0.6 mg.

In another specific embodiment, vitamin B1 may be included in specific ranges or amounts for each specific form. When provided in their specific forms, the provided numerical range or amount includes the amounts of the specific form and/or compounds that are equivalent to the specific form.

The compositions, kits and methods of the present invention may comprise or use vitamin B2. Vitamin B2 is a component of two flavin coenzymes, flavin mononucleotide (FMN) and flavin adenine dinucleotide (FAD). These flavoenzymes are involved in a number of oxidation-reduction reactions including the conversion of pyridoxine and niacin. Flavoenzymes also play a role in a number of metabolic pathways such as amino acid deamination, purine degradation and fatty acid oxidation and thus help to maintain carbohydrate, amino acid and lipid metabolism.

In a specific embodiment, vitamin B2 may be in one or more of the forms of flavin mononucleotide (FMN), flavin adenine dinucleotide (FAD), riboflavin (also known as 7,8-dimethyl-10-((2R,3R,4S)-2,3,4,5-tetrahydroxypentyl)benzo[g]pteridine-2,4 (3H,10H)-dione or lactoflavin) and riboflavin derivatives such as riboflavin-5′-monophosphate, riboflavin-5′-monobutyrate and riboflavin-5′-monopalmitate. In a specific embodiment of the present invention, vitamin B2 may be included in the form of riboflavin

In another specific embodiment, vitamin B2 may be included in amounts ranging from about 0.2 mg to about 1.0 mg. In another specific embodiment, vitamin B2 is riboflavin between 0.4 and 0.6 mg.

The compositions, kits and methods of the present invention may comprise or use vitamin B3. Vitamin B3, or “niacin” is the common name for two compounds: nicotinic acid (also called niacin) and niacinamide (also called nicotinamide). Vitamin B3 is particularly important for maintaining healthy levels and types of fatty acids. It is also required for the synthesis of pyroxidine, riboflavin, and folic acid. RDA, supra at 137. Administration of vitamin B3 also may effect a reduction in total cholesterol (LDL) and very low density lipoprotein (VLDL) levels and an increase in high density lipoprotein (HDL) cholesterol levels. Nicotinamide adenine dinucleotide (NAD) and NAD phosphate (NADP) are active coenzymes of niacin. These coenzymes are involved in numerous enzymatic reactions such as glycolysis, fatty acid metabolism, and steroid synthesis. Henkin et al., 91 AM. J. MED. 239-46 (1991). In a specific embodiment, vitamin B3 is nicotinamide or salts and esters thereof at a range between 2 to 20 mg, and specifically 5 to 7 mg.

The compositions for the invention include a water-soluble vitamin B5. Pantothenic acid is an essential nutrient. Animals require pantothenic acid to synthesize coenzyme-A (CoA), as well as to synthesize and metabolize proteins, carbohydrates, and fats. Pantothenic acid is the amide between pantoic acid and f3-alanine. In one specific embodiment, Vitamin B5 is D-Calcium Pantothenate in an amount between 0.5 and 3.0 mg, and specifically between 1.5 to 2.5 mg.

The compositions of the invention may include vitamin B6. The administration of vitamin B6 may reduce the levels of homocysteine. Bostom et al., 49 KIDNEY INT. 147-52 (1996). The active forms of vitamin B6, pyridoxal-5′-phosphate (PLP) and pyridoxamine-5′-phosphate, are coenzymes for numerous enzymes and as such, are important for gluconeogenesis, niacin formation, and erythrocyte metabolism. RDA, supra at 142-43. Vitamin B6 is a coenzyme for both cystathionine synthase and cystathionase, enzymes that catalyze the formation of cysteine from methionine. Homocysteine is an intermediate in this process and elevated levels of plasma homocysteine are recognized as a risk factor for both vascular disease (Robinson et al., 94 CIRCULATION 2743-48 (1996)) and neural tube defects (Locksmith & Duff, 91 OB STET. GYNECOL. 1027-34 (1998)). In a specific embodiment, vitamin B6 is may be included in the forms of pyridoxine hydrochloride in a range of 0.1 to 1.0 mg, and specifically 0.5 to 0.7 mg. In another specific embodiment, vitamin B5 or B6 may be included in specific ranges or amounts for each specific form. When provided in their specific forms, the provided numerical range or amount includes the amounts of the specific form and/or compounds that are equivalent to the specific form.

The compositions, kits and methods of the present invention may comprise or use vitamin Folate (Vitamin B9). The term folate, which itself is the generic name for many different forms of water-soluble vitamin species is essential for DNA synthesis and, hence, cell division. Simpson et al., THE JOURNAL OF MATERNAL-FETAL AND NEONATAL MEDICINE, Micronutrients and women of reproductive potential: required dietary intake and consequences of dietary deficiency or excess. Folate encompasses numerous compounds that for example, are based on a pteridine ring, an aminobenzoic acid and one or more glutamic acid residues. Folic acid (pteroglutamic acid or PGA) is a synthetic form of folate, and the first folate synthesized and used as a supplement. The term folates may also be used in the generic sense to designate any members of the family of pteroylglutamates, or mixtures of them, having various levels of reduction of the pteridine ring, one-carbon substitutions and numbers of glutamate residues. PURE & APPL. CHEM., IUPAC-IUB Commission on Biochemical Nomenclature (CBN). Nomenclature and Symbols for Folic Acid and Related Compounds. Arch 59, No. 6: 833-836 (1987).

Vitamin may be defined by its structure, and also by various functions. Indeed, vitamin B9 is essential for DNA synthesis and, hence, cell division and is required metabolically as a coenzyme in one-carbon transfer reactions. Folic acid, a commonly used term synonymous with vitamin B9, is known to reduce the risk of multiple diseases. Clinical trials definitively demonstrated the effectiveness of folic acid supplementation in reducing the number of neural tube defects. In a specific embodiment, Folate is included as Folic Acid and is present in an amount between 50 mcg to 300 mcg, and specifically between 125 mcg to 175 mcg.

In another embodiment, vitamin B12 may be included in amounts ranging from about 6 μg to about 18 μg. In another specific embodiment, vitamin B12 may be included in amounts ranging from about 9.6 μg to about 14.4 μg. In another specific embodiment, vitamin B12 may be included in amounts ranging from about 10.8 μg to about 13.2 μg. In another embodiment, vitamin B12 may be included in an amount of about 12 μg.

In another specific embodiment, vitamin B12 may be included in specific ranges or amounts for each specific form. For example, vitamin B12 may be in the form cyanocobalamin and may be included in the amount of about 0.5 to 1.5 mcg, and specifically between 0.7 to 0.9 mcg.

The compositions of the present invention may comprise or use vitamin C. The major biochemical role of water-soluble vitamin C is as a co-substrate in metal catalyzed hydroxylations. Vitamin C has antioxidant properties and interacts directly with superoxide hydroxyl radicals and singlet oxygen, and also provides antioxidant protection for folate and vitamin E, keeping vitamin E in its most potent form. Vitamin C enhances the absorption of iron. In addition, vitamin C is required for collagen synthesis, epinephrine synthesis, and bile acid formation. In a specific embodiment of the present invention, vitamin C may be included in the forms of ascorbic acid, ascorbates (calcium or sodium ascorbate), dehydroascorbic acid and salts, ascorbyl palmitate, ascorbyl phosphates and salts (such as sodium or magnesium ascorbyl phosphate), ascorbyl tetraisopalmitate, tetrahexyldecyl ascorbate, ascorbyl sulfates and salts, acylated ascorbic acid derivatives (such as 6-O-acyl-2-O-alpha-D-glucopyranosyl-L-ascorbic acids), 6-bromo-6-deoxy-L-ascorbic acid, and ascorbate salts. In a specific embodiment, vitamin C may be included in the form of ascorbic acid in an amount between 10 and 50 mh and specifically between 20-30 mg.

The compositions of the present invention may comprise or use vitamin D. In another embodiment, the compositions and methods of the present invention may include a beneficially increased supplementation of vitamin D. Vitamin D is a fat-soluble “hormone like” substance important for the maintenance of healthy bones. This vitamin increases the absorption of calcium and phosphorous from the gastrointestinal tract and improves mineral resorption into bone tissue.

The vitamin D of the compositions of the present invention may comprise vitamin D in one or more the forms of vitamin D3 (also known as calciol or cholecalciferol or colecalciferol), vitamin D2 (also known as calciferol, ergocalciol, ergocalciferol, ercalciol, Deltalin or Viosterol), previtamin D2, ergosterol, calcitriol (also known as 1,25-dihydroxycholecalciferol), 7-dehydrocholesterol, vitamin D1, vitamin D4 (also known as 22-dihydroergocalciferol, 22,23-dihydroercalciol or (24S)-methylcalciol), vitamin D5 (also known as (24S)-Ethylcalciol or sitocalciferol), 7-dehydrositosterol, Lumisterol, 25-hydroxyvitamin D, all steroids that exhibit the biological activity of calciol, 25-fluorocalciol, (3S)-3-amino-3-deoxycalciol, 11α-acetoxycalciol, calcidiol (also known as 25-hydroxycholecalciferol or calcifediol), ercalcitriol, calcitetrol, tacalciol (also known as tachysterol3), (5E)-isocalciol (also known as isovitamin D3), Dihydroercalciol (also known as dihydrotachysterol3), (1S)-Hydroxycalciol (also known as 1α-hydroxycholecalciferol or alfacaleidol), (24R)-Hydroxycalcidiol (also known as 24(R),25-dihydroxycholecalciferol), Ercalcidiol, Ercalcitriol, Ertacalciol, (5E)-(10S)-10,19-Dihydroercalciol (also known as dihydrotachysterol2), (6Z)-Tacalciol (also known as precalciferol or pre-vitamin D), and (22E)-(24R)-Ethyl-22,23-didehydrocalciol also known as vitamin D6. In one embodiment of the invention, vitamin D is in the form of Cholecalciferol in an amount between 50 and 500 IU, and specifically between 150 to 250 IU.

The compositions, kits and methods of the present invention may comprise or use vitamin E. Vitamin E is a fat-soluble vitamin antioxidant found in biological membranes where it protects the phospholipid membrane from oxidative stress. Vitamin E inhibits the oxidation of unsaturated fatty acids by trapping peroxyl free radicals. It is also an antiatherogenic agent, and studies have demonstrated a reduced risk of coronary heart disease with increased intake of vitamin E. In a specific embodiment, vitamin E may be included in one or more of the forms of alpha, beta, gamma, and delta tocopherols in its natural or synthetic (dl) forms; alpha, beta, gamma, and delta tocotrienols in its natural or synthetic (dl) forms, dl-alpha tocopheryl derivatives such as dl-alpha tocopheryl esters, dl-alpha-tocopheryl acetate or succinate and d-alpha-tocopheryl acetate or dl-alpha tocopheryl phosphates (such as Ester-E®). In a specific embodiment of the present invention, vitamin E may be included in the form of d-alpha-tocopheryl acetate between 1-20 IU and specifically between 5 and 10 IU.

The compositions of the present invention may comprise iron. A primary function of iron is to carry oxygen to bodily tissues via the hemoglobin part of red blood cells. Supplemental intake of iron is critical to preventing anemia, a disorder associated with a variety of physiological states. The, compositions, kits and methods of the present invention may include iron in one or more of the forms of elemental iron, in the form of a salt, chelated form, non-chelated form, chelated to an amino acid, carbonyl iron, ferrous gluconate, ferrous fumarate, polysaccharide iron complex, elemental polysaccharide iron, polysaccharide iron, ferrous (II)-bis-glycinate chelate, ferrous asparto glycinate, ferrous bisglycinate, ferrous bisglycinate hydrochloride, ferrous bisglycinate, elemental ferrous bisglycinate, ferrous sulfate, ferronyl (micronized), iron protein succinylate, carbonyl iron, Sumalate iron, Heme iron complex, as Ferrochel amino acid chelate, heme iron polypeptide as Proferrin-bovine source, as heme iron polypeptide (bovine source) as sodium iron EDTA (Ferrazone), ferric ammonium citrate, elemental iron, ferric pyrophosphate, and Ferrous Lactate. In one embodiment, the iron is Ferrous Lactate in an amount between 5-10 mg, and specifically 7-8 mg.

The compositions of the present invention may comprise calcium, preferably calcium carbonate in a range of between 10-50 mg, and specifically between 20-30 mg.

The compositions of the present invention may comprise iodine. Iodine provides nutritional benefits as it is an essential component of the thyroid hormones that are involved in the regulation of various enzymes and metabolic processes, such as thyroxine and triiodothyronine. Thyroid hormones play pivotal roles in metabolism. In a specific embodiment, iodine may be in the forms of elemental iodine, iodized salt, Lugol's iodine, sodium iodide, potassium iodide, potassium iodate, nascent iodine, and Nano-Colloidal Detoxified Iodine. In a specific embodiment, iodine is Potassium Iodide at between 10 and 100 mcg, and specifically 25-75 mcg.

The compositions of the present invention comprise magnesium. Magnesium is found primarily in both bone and muscle and is important for over 300 different enzyme reactions. A primary function of magnesium is to bind to phosphate groups in adenosine triphosphate (ATP), thereby forming a complex that assists in the transfer of ATP phosphate. Magnesium also functions within cells as a membrane stabilizer. Magnesium plays roles in nucleic acid synthesis, glycolysis, transcription of DNA and RNA, amino acid activation, membrane transport, transketolase reactions, and protein synthesis. It is also involved in the formation of cAMP, a cytosolic second messenger that plays a role in cell signaling mechanisms. Magnesium also functions both synergistically and antagonistically with calcium in neuromuscular transmission. Specifically, magnesium is critical for the maintenance of electrochemical potentials of nerve and muscle membranes and the neuromuscular junction transmissions, particularly important in the heart.

Magnesium is available in a variety of salts and can be included in either chelated or nonchelated form. In one specific embodiment of the present invention, magnesium may be included in the forms of elemental magnesium, in the form of a salt, in a chelated form, in a non-chelated form, magnesium acetate, magnesium carbonate, magnesium oxide, magnesium gluconate, magnesium chloride, magnesium citrate, magnesium silicate, magnesium stearate, magnesium sulfate, magnesium oxide, and magnesium chelated to an amino acid (magnesium glycinate, magnesium aspartate). In a specific embodiment, magnesium may be magnesium oxide present in the amounts ranging from about 2.5 mg to about 7.5 mg. In another embodiment, magnesium oxide may be present specifically in the amounts ranging from about 25 to about 35 mcg.

The compositions of the invention comprise zinc. Zinc plays a role in numerous metabolic activities such as nucleic acid production, protein synthesis, and development of the immune system. There are more than 200 zinc metalloenzymes including aldolase, alcohol dehydrogenase, RNA polymerase, and protein kinase C. Zinc stabilizes RNA and DNA structures, forms zinc fingers in nuclear receptors, and is a component of chromatin proteins involved in transcription and replication.

In a specific embodiment of the invention, zinc may be provided in one or more of the forms of elemental zinc, in the form of a salt, in a chelated form, in a non-chelated form, zinc acetate, zinc gluconate, zinc picolinate, zinc sulfate and zinc oxide. In a specific embodiment of the present invention, zinc may be included in the form of zinc oxide. In another specific embodiment, zinc is zinc gluconate in an amount between 5 and 10 mg, and specifically between 7-8 mg.

The compositions of the present invention comprise omega-3 fatty acids. Omega-3 fatty acids play integral roles in physiological mechanisms that serve to prevent, treat and/or alleviate the occurrence or negative effects of some diseases and has shown multiple health-promoting properties in adults. For example, omega-3 fatty acids are linked to health benefits such as preventing the occurrence of cancer, preventing the occurrence of heart disease, and are helpful in brain health and immune function. Indeed, omega-3 fatty acids include essential fatty acids linked to numerous health benefits, such as docahexaenoic acid (or docosahexaenoic acid, DHA) and eicosapentaenoic acid (EPA. In a specific embodiment, the compositions of the present invention comprise Docahexaenoic acid (or docosahexaenoic acid, DHA) and eicosapentaenoic acid (EPA).

DHA may be obtained in solid form, such as in a whole-cell microbial product, or in liquid form, such as in an oil. In a specific embodiment of the present invention, the source of DHA and EPA is Omega-3 powder.

In another specific embodiment, omega-3 fatty acids may be included in specific ranges or amounts for each specific form. When provided in their specific forms, the provided numerical range or amount includes the amounts of the specific form and/or compounds that are equivalent to the specific form. For example, omega-3 fatty acids may be in the form of DHA and may be included in the amount of about 10 to about 50 mg and specifically 20-25 mg and EPA between about 5-10 μm and specifically 7-8 mg EPA. In combinations, the total of DHA and EPA can vary between 15 and 50 mg and comprise individual fractions of each between these ranges.

The compositions of the invention comprise phosphatidylserine. Phosphatidylserine or 1,2-diacyl-sn-glycero-3-phospho-L-serine is an important anionic phospholipid, which brings essential physical properties to membranes in both eukaryotes and prokaryotes. Independently of this, it has many biological functions in cells, including effects on blood coagulation and apoptosis, and it is the precursor for phosphatidylethanolamine in prokaryotes and in eukaryote mitochondria. Its metabolite lysophosphatidylserine has signaling functions and operates through specific receptors. Also, there is increasing interest in a structurally related lipid phosphatidylthreonine and other phospholipids linked to amino acids.

Phosphatidylserine is an acidic (anionic) phospholipid with three ionizable groups, i.e. the phosphate moiety, the amino group and the carboxyl function. As with other acidic lipids, it exists in nature in salt form, but it has a high propensity to chelate to calcium via the charged oxygen atoms of both the carboxyl and phosphate moieties, modifying the conformation of the polar head group. This interaction may be of considerable relevance to the biological function of phosphatidylserine, especially during bone formation for example. In a specific embodiment, the composition includes between 5 and 50 mg, and specifically between 15-25 mg.

Selemium is provided in the form of a selenium yeast as described in U.S. Pat. No. 6,368,643 which is specifically incorporated by reference. Selenium yeast is present in an amount ranging from 5-50 mcg and specifically 15-25 mcg.

The compositions of the present invention comprise Lutein from Marigolf Flower. Apart from its ornamental value, Marigold (Tagetes erecta L.) flowers are well known as an herbal remedy due to its antimicrobial, anti-inflammatory, and anti-oxidant activities. Epidemiological studies have implicated prolonged exposure to ultraviolet radiations & blue light and in turn oxidative stress in the pathogenesis of the majority of the eye diseases, since childhood. Studies have shown that with age a number of changes occur predisposing the retinal various organs and tissues to oxidative stress. These changes manifest in decreased levels in plasma of Vitamin C, Vitamin E, glutathione, Retinal Pigment Epithelium (RPE), Catalase (CAT), Super Oxide Dismutase (SOD), Thiobarbituric Acid Reactive Substance (TBARS), and total anti-oxidant capacity (TAC). Age- and diet-related loss of Lutein and Zeaxanthin enhance phototoxic damage to the eye, and thus supplementation of these carotenoids becomes vital for maintaining optimal eye health. In a specific embodiment of the present invention, Lutein is provided in a Marifold extract, for example at 5% lutein in an amount between 25 and 100 mg and specifically between 55-70 mg.

Choline is used in the synthesis of certain phospholipids (phosphatidylcholine and sphingomyelin) that are essential structural components of cell membranes. Phosphatidylcholine accounts for about 95% of total choline in tissues. This phospholipid can be synthesized from dietary choline via the cytidine diphosphocholine (CDP-choline) pathway or through the methylation of another phospholipid, phosphatidylethanolamine. Sphingomyelin is a type of sphingosine-containing phospholipid (sphingolipid) that is synthesized by the transfer of a phosphocholine residue from a phosphatidylcholine to a ceramide. Sphingomyelin is found in cell membranes and in the fatty sheath that envelops myelinated nerve fibers. In a specific embodiment of the present invention, choline is Choline Bitartate in an amount between 20 and 100 mg and specifically between 25-75 mg.

The first example of the invention is comprised of:
1. 800 IU to 900 IU of Vitamin A in the form of Vitamin A Palmitate
2. 20 mg to 30 mg of Vitamin C in the form of Ascorbic Acid
3. 150 IU to 250 IU of Vitamin D in the form of Cholecalciferol
4. 5 IU to 10 IU of Vitamin E in the form of D-Alpha Tocopheryl Acetate
5. 0.4 mg to 0.6 mg of Vitamin B1 in the form of Thiamine Hydrochloride
6. 0.4 mg to 0.6 mg of Vitamin B2 in the form of Riboflavin
7. 5 mg to 7 mg of Vitamin B3 in the form of Nicotinamide
8. 1.5 mg to 2.5 mg of Vitamin B5 in the form of D-Calcium Pantothenate
9. 0.5 mg to 0.7 mg of Vitamin B6 in the form of Pyridoxine Hydrochloride
10. 5 mcg to 15 mcg of Biotin in the form of D-Biotin
11. 125 mcg to 175 mcg of Folate in the form of Folic Acid
12. 0.7 mcg to 0.9 mcg of Vitamin B12 in the form of Cyanocobalamin
13. 25 mcg to 75 mcg of Iodine in the form of Potassium Iodide
14. 15 mcg to 25 mcg of Selenium in the form of Selenium Yeast
15. 50 mg to 70 mg of Marigold Flower containing 5% Lutein
16. 25 mg to 75 mg of Choline in the form of Choline Bitartrate
17. 7 mg to 8 mg of Iron in the form of Ferrous Lactate
18. 15 mg to 25 mg of Phosphatidylserine
19. 20 mg to 25 mg of DHA from Omega-3 powder
20. 7 mg to 8 mg of EPA from Omega-3 powder
21. 20 mg to 30 mg of Calcium in the form of Calcium Carbonate
22. 25 mg to 35 mg of Magnesium in the form of Magnesium Oxide
23. 7 mg to 8 mg of Zinc in the form of Zinc Gluconate
24. 1000 mg to 1500 mg of Sucrose
25. 100 mg to 500 mg of Flavoring

A single dose will be taken orally and will be between 2000 mg to 3000 mg. The nutritional supplement in powder form may contain additional ingredients and is not limited to the present ingredients listed.

Each of these ingredients listed in the example are commercially available from many suppliers with the exception of the microencapsulated fish oil powder that is only available from a select few. The process in which the microencapsulated fish oil powder is produced is as follows: (1) The fish oil is standardized to a ratio of 7% DHA and 1.5% EPA (2) The oil is encapsulated into micro spheres using a starch based powder (3) The microencapsulation is complete when the starch completely encapsulates the fish oil (4) The packaged fish oil powder is ready for commercial use.

The manufacturing process for the first example is as follows: (1) Weigh out each ingredient precisely (2) Combine ingredients numbered 1-13 into a blender or mixing vessel (3) Blend ingredients 1-13 for five minutes (4) Combine ingredients numbered 14-19 into a blender or mixing vessel (4) Blend ingredients 14-20 for five minutes (5) Combine ingredients numbered 21-23 into a blender or mixing vessel (6) Blend ingredients 21-23 for five minutes (7) Combine ingredients numbered 24-25 into a blender or mixing vessel (8) Blend ingredients 24-25 for five minutes (9) Combine all ingredients into a blender or mixing vessel (10) Blend all ingredients for five minutes (11) Finished product is packaged for consumption.

The second example of the invention is comprised of:
1. 800 IU to 900 IU of Vitamin A in the form of Vitamin A Palmitate
2. 20 mg to 30 mg of Vitamin C in the form of Ascorbic Acid
3. 150 IU to 250 IU of Vitamin D in the form of Cholecalciferol
4. 5 IU to 10 IU of Vitamin E in the form of D-Alpha Tocopheryl Acetate
5. 0.4 mg to 0.6 mg of Vitamin B1 in the form of Thiamine Hydrochloride
6. 0.4 mg to 0.6 mg of Vitamin B2 in the form of Riboflavin
7. 5 mg to 7 mg of Vitamin B3 in the form of Nicotinamide
8. 1.5 mg to 2.5 mg of Vitamin B5 in the form of D-Calcium Pantothenate
9. 0.5 mg to 0.7 mg of Vitamin B6 in the form of Pyridoxine Hydrochloride
10. 5 mcg to 15 mcg of Biotin in the form of D-Biotin
11. 125 mcg to 175 mcg of Folate in the form of Folic Acid
12. 0.7 mcg to 0.9 mcg of Vitamin B12 in the form of Cyanocobalamin
13. 25 mcg to 75 mcg of Iodine in the form of Potassium Iodide
14. 15 mcg to 25 mcg of Selenium in the form of Selenium Yeast
15. 50 mg to 70 mg of Marigold Flower containing 5% Lutein
16. 25 mg to 75 mg of Choline in the form of Choline Bitartrate
17. 7 mg to 8 mg of Iron in the form of Ferrous Lactate
18. 15 mg to 25 mg of Phosphatidylserine
19. 20 mg to 25 mg of DHA from Omega-3 powder
20. 7 mg to 8 mg of EPA from Omega-3 powder
21. 20 mg to 30 mg of Calcium in the form of Calcium Carbonate
22. 25 mg to 35 mg of Magnesium in the form of Magnesium Oxide
23. 7 mg to 8 mg of Zinc in the form of Zinc Gluconate
24. 100 mg to 500 mg of Dextrose
25. 10 mg to 50 mg of Flavoring

A single dose will be taken orally and will be between 1000 mg to 1600 mg. The nutritional supplement in tablet form may contain additional ingredients and is not limited to the present ingredients listed.

Each of these ingredients listed in the example are commercially available from many suppliers with the exception of the microencapsulated fish oil powder that is only available from a select few. The process in which the microencapsulated fish oil powder is produced is as follows: (1) The fish oil is standardized to a ratio of 7% DHA and 1.5% EPA (2) The oil is encapsulated into micro spheres using a starch based powder (3) The microencapsulation is complete when the starch completely encapsulates the fish oil (4) The packaged fish oil powder is ready for commercial use.

The manufacturing process for the second example is as follows: (1) Weigh out each ingredient precisely (2) Combine ingredients numbered 1-13 into a blender or mixing vessel (3) Blend ingredients 1-13 for five minutes (4) Combine ingredients numbered 14-19 into a blender or mixing vessel (4) Blend ingredients 14-20 for five minutes (5) Combine ingredients numbered 21-23 into a blender or mixing vessel (6) Blend ingredients 21-23 for five minutes (7) Combine ingredients numbered 24-25 into a blender or mixing vessel (8) Blend ingredients 24-25 for five minutes (9) Combine all ingredients into a blender or mixing vessel (10) Blend all ingredients for five minutes (11) Tablets are produced from the ingredients blend (12) Finished product is packaged for consumption.

It should be noted that all features, elements, components, functions, and steps described with respect to any embodiment provided herein are intended to be freely combinable and substitutable with those from any other embodiment. If a certain feature, element, component, function, or step is described with respect to only one embodiment, then it should be understood that that feature, element, component, function, or step can be used with every other embodiment described herein unless explicitly stated otherwise. This paragraph therefore serves as antecedent basis and written support for the introduction of claims, at any time, that combine features, elements, components, functions, and steps from different embodiments, or that substitute features, elements, components, functions, and steps from one embodiment with those of another, even if the following description does not explicitly state, in a particular instance, that such combinations or substitutions are possible. It is explicitly acknowledged that express recitation of every possible combination and substitution is overly burdensome, especially given that the permissibility of each and every such combination and substitution will be readily recognized by those of ordinary skill in the art.

While the embodiments are susceptible to various modifications and alternative forms, specific examples thereof are herein described in detail. It should be understood, however, that these embodiments are not to be limited to the particular form disclosed, but to the contrary, these embodiments are to cover all modifications, equivalents, and alternatives falling within the spirit of the disclosure. Furthermore, any features, functions, steps, or elements of the embodiments may be recited in or added to the claims, as well as negative limitations that define the inventive scope of the claims by features, functions, steps, or elements that are not within that scope.

Other compositions and advantages of the subject matter described herein will be or will become apparent to one with skill in the art upon examination of the foregoing description. In no way should the features of the example embodiments be construed as limiting the appended claims, absent express recitation of those features in the claims.

1. An orally administered nutritional composition comprising:
a vitamin selected form the group consisting of: Vitamin A in the form of Vitamin A Palmitate; Vitamin C in the form of Ascorbic Acid; Vitamin D in the form of Cholecalciferol; of Vitamin E in the form of D-Alpha Tocopheryl Acetate; Vitamin B1 in the form of Thiamine Hydrochloride; Vitamin B2 in the form of Riboflavin; Vitamin B3 in the form of Nicotinamide; Vitamin B5 in the form of D-Calcium Pantothenate; Vitamin B6 in the form of Pyridoxine Hydrochloride; Biotin in the form of D-Biotin; Folate in the form of Folic Acid; Vitamin B12 in the form of Cyanocobalamin and combinations thereof;
a chemical element selected from the group consisting of Iodine as potassium iodide, Selenium as Selenum Yeast, Calcium as Calcium Carbonate, Magnesium as Magnesium Chloride, and Zinc as Zinc gluconate, iron in the form of ferrous lactate, and combinations thereof; and
a nutraceutical substance selected from the group consisting of lutein, Choline in the form of choline bitartrate, phoshpatidyl serine, microencapsulated docosahexaenoic acid (DHA) and eicosapentaenoic acid (EPA) from Omega 3 powder, and combinations thereof.

2. The nutritional composition of claim 1, comprising 800 IU to 900 IU of the Vitamin A in the form of Vitamin A Palmitate, 20 mg to 30 mg of the Vitamin C in the form of Ascorbic Acid, 150 IU to 250 IU of the Vitamin D in the form of Cholecalciferol, and 5 IU to 10 IU of the Vitamin E in the form of D-Alpha Tocopheryl Acetate.

3. The nutritional composition of claim 1, comprising 0.4 mg to 0.6 mg of the Vitamin B1 in the form of Thiamine Hydrochloride; 0.4 mg to 0.6 mg of the Vitamin B2 in the form of Riboflavin, 5 mg to 7 mg of the Vitamin B3 in the form of Nicotinamide, 1.5 mg to 2.5 mg of the Vitamin B5 in the form of D-Calcium Pantothenate, and 0.5 mg to 0.7 mg of Vitamin B6 in the form of Pyridoxine Hydrochloride.

4. The nutritional composition of claim 1, comprising 5 mcg to 15 mcg of the Biotin in the form of D-Biotin, 125 mcg to 175 mcg of the Folate in the form of Folic Acid, and 0.7 mcg to 0.9 mcg of the Vitamin B12 in the form of Cyanocobalamin.

5. The nutritional composition of claim 1, comprising 25 mcg to 75 mcg of the Iodine in the form of Potassium Iodide, 15 mcg to 25 mcg of the Selenium in the form of Selenium Yeast, 20 mg to 30 mg of the Calcium in the form of Calcium Carbonate, 25 mg to 35 mg of the Magnesium in the form of Magnesium Oxide, 7 mg to 8 mg of the Zinc in the form of Zinc Gluconate, and 7 mg to 8 mg of Iron in the form of Ferrous Lactate.

6. The nutritional composition of claim 1, comprising 50 mg to 70 mg of the lutein as a Marigold Flower derivative containing approximately 5% Lutein, 25 mg to 75 mg of the Choline in the form of Choline Bitartrate, 15 mg to 25 mg of the Phosphatidylserine, and 10 mg to 25 mg of DHA and 7 mg to 8 mg of EPA from Omega-3 powder.

7. The nutritional composition of claim 1 further comprising dextrose or sucrose.

8. The nutritional composition of claim 7, comprising 100 to 500 mg of dextrose or 1000 to 1500 mg of sucrose.

9. The nutritional composition of claim 1, comprising microencapsulated docosahexaenoic acid (DHA) and eicosapentaenoic acid (EPA) from Omega 3 powder between 15 and 50 mg in combination, and combinations thereof, 800 IU to 900 IU of the Vitamin A in the form of Vitamin A Palmitate, 20 mg to 30 mg of the Vitamin C in the form of Ascorbic Acid, 150 IU to 250 IU of the Vitamin D in the form of Cholecalciferol, and 5 IU to 10 IU of the Vitamin E in the form of D-Alpha Tocopheryl Acetate.

10. The nutritional composition of claim 1, comprising microencapsulated docosahexaenoic acid (DHA) and eicosapentaenoic acid (EPA) from Omega 3 powder between 15 and 50 mg in combination, 0.4 mg to 0.6 mg of the Vitamin B1 in the form of Thiamine Hydrochloride; 0.4 mg to 0.6 mg of the Vitamin B2 in the form of Riboflavin, 5 mg to 7 mg of the Vitamin B3 in the form of Nicotinamide, 1.5 mg to 2.5 mg of the Vitamin B5 in the form of D-Calcium Pantothenate, and 0.5 mg to 0.7 mg of Vitamin B6 in the form of Pyridoxine Hydrochloride.

11. The nutritional composition of claim 1, comprising microencapsulated docosahexaenoic acid (DHA) and eicosapentaenoic acid (EPA) from Omega 3 powder between 15 and 50 mg in combination, 5 mcg to 15 mcg of the Biotin in the form of D-Biotin, 125 mcg to 175 mcg of the Folate in the form of Folic Acid, and 0.7 mcg to 0.9 mcg of the Vitamin B12 in the form of Cyanocobalamin.

12. The nutritional composition of claim 1, comprising microencapsulated docosahexaenoic acid (DHA) and eicosapentaenoic acid (EPA) from Omega 3 powder between 15 and 50 mg in combination, 25 mcg to 75 mcg of the Iodine in the form of Potassium Iodide, 15 mcg to 25 mcg of the Selenium in the form of Selenium Yeast, 20 mg to 30 mg of the Calcium in the form of Calcium Carbonate, 25 mg to 35 mg of the Magnesium in the form of Magnesium Oxide, 7 mg to 8 mg of the Zinc in the form of Zinc Gluconate, and 7 mg to 8 mg of Iron in the form of Ferrous Lactate.

13. The nutritional composition of claim 1, comprising microencapsulated docosahexaenoic acid (DHA) and eicosapentaenoic acid (EPA) from Omega 3 powder between 15 and 50 mg in combination, 50 mg to 70 mg of the lutein as a Marigold Flower derivative containing approximately 5% Lutein, 25 mg to 75 mg of the Choline in the form of Choline Bitartrate, 15 mg to 25 mg of the Phosphatidylserine, and 10 mg to 25 mg of DHA and 7 mg to 8 mg of EPA from Omega-3 powder.

14. A method to produce a supplement having Omega 3 constituents' docosahexaenoic acid (DHA) and eicosapentaenoic acid (EPA) comprising:
1) blending microencapsulated docosahexaenoic acid (DHA) and eicosapentaenoic acid (EPA) from Omega 3 powder in combination with:
a vitamin selected form the group consisting of: Vitamin A in the form of Vitamin A Palmitate; Vitamin C in the form of Ascorbic Acid; Vitamin D in the form of Cholecalciferol; of Vitamin E in the form of D-Alpha Tocopheryl Acetate; Vitamin B1 in the form of Thiamine Hydrochloride; Vitamin B2 in the form of Riboflavin; Vitamin B3 in the form of Nicotinamide; Vitamin B5 in the form of D-Calcium Pantothenate; Vitamin B6 in the form of Pyridoxine Hydrochloride; Biotin in the form of D-Biotin; Folate in the form of Folic Acid; Vitamin B12 in the form of Cyanocobalamin and combinations thereof;
a chemical element selected from the group consisting of Iodine as potassium iodide, Selenium as Selenum Yeast, Calcium as Calcium Carbonate, Magnesium as Magnesium Chloride, and Zinc as Zinc gluconate, iron in the form of ferrous lactate, and combinations thereof; and
a nutraceutical substance selected from the group consisting of lutein, choline in the form of choline bitartrate, and phoshpatidyl serine; and
2) producing a dose of the supplement in tablet form.

15. The method of claim 14, wherein the microencapsulated docosahexaenoic acid (DHA) and eicosapentaenoic acid (EPA) from Omega 3 powder is produced from fish oil.

16. The method of claim 15, wherein the microencapsulated docosahexaenoic acid (DHA) and eicosapentaenoic acid (EPA) from Omega 3 powder from fish oil is produced by microencapsulation in microspheres using a starch base powder.

17. The method of claim 14, wherein the supplement comprises:
800 IU to 900 IU of the Vitamin A in the form of Vitamin A Palmitate, 20 mg to 30 mg of the Vitamin C in the form of Ascorbic Acid, 150 IU to 250 IU of the Vitamin D in the form of Cholecalciferol, and 5 IU to 10 IU of the Vitamin E in the form of D-Alpha Tocopheryl Acetate, 0.4 mg to 0.6 mg of the Vitamin B1 in the form of Thiamine Hydrochloride; 0.4 mg to 0.6 mg of the Vitamin B2 in the form of Riboflavin, 5 mg to 7 mg of the Vitamin B3 in the form of Nicotinamide, 1.5 mg to 2.5 mg of the Vitamin B5 in the form of D-Calcium Pantothenate, 0.5 mg to 0.7 mg of Vitamin B6 in the form of Pyridoxine Hydrochloride, and 5 mcg to 15 mcg of the Biotin in the form of D-Biotin, 125 mcg to 175 mcg of the Folate in the form of Folic Acid, and 0.7 mcg to 0.9 mcg of the Vitamin B12 in the form of Cyanocobalamin.

18. The method of claim 14, wherein the supplement comprises:
25 mcg to 75 mcg of the Iodine in the form of Potassium Iodide, 15 mcg to 25 mcg of the Selenium in the form of Selenium Yeast, 20 mg to 30 mg of the Calcium in the form of Calcium Carbonate, 25 mg to 35 mg of the Magnesium in the form of Magnesium Oxide, 7 mg to 8 mg of the Zinc in the form of Zinc Gluconate, and 7 mg to 8 mg of Iron in the form of Ferrous Lactate.

19. The method of claim 14, wherein the supplement comprises:
50 mg to 70 mg of the lutein as a Marigold Flower derivative containing approximately 5% Lutein, 25 mg to 75 mg of the Choline in the form of Choline Bitartrate, 15 mg to 25 mg of the Phosphatidylserine, and 10 mg to 25 mg of DHA and 7 mg to 8 mg of EPA from Omega-3 powder.
